(19) United States
(12) Patent Application Publication
(10) Pub. No.: US 2020/0115712 A1
(43) Pub. Date: Apr. 16, 2020

(54) COMPOSITIONS AND METHODS FOR CANCER IMMUNOTHERAPY

(71) Applicant: Silenseed Ltd., Modi'in (IL)

(72) Inventors: Amotz Shemi, Modi'in (IL); Rachel Malka Gabai, Modi'in (IL); Shay Rotkopf, Modi'in (IL)

(73) Assignee: Silenseed Ltd., Modi'in (IL)

(21) Appl. No.: 16/616,464

(22) PCT Filed: May 23, 2018
(86) PCT No.: PCT/IL2018/050560

§ 371 Date: Nov. 24, 2019

Related U.S. Application Data
(60) Provisional application No. 62/510,281, filed on May 24, 2017.

(51) Int. Cl. C12N 15/113; C07K 16/28; A61P 35/00; A61K 47/34

(52) U.S. Cl. CPC C12N 15/1135; C07K 16/2827; C07K 16/2818; A61K 2039/507; A61K 47/34; C07K 2317/76; A61P 35/00

(57) ABSTRACT

Provided herein are treatments for improving cancer immunotherapy, and particularly in solid tumors. The described treatments include sustained release oligonucleotide agents, optionally together with immunotherapy agents. Methods of treating cancer with the described treatments are also disclosed.

### CROSS REFERENCE TO RELATED APPLICATIONS

Benefit is claimed to U.S. Provisional Patent Application No. 62/510,281, filed May 24, 2017, the contents of which are incorporated by reference herein in their entirety.

### FIELD

This disclosure relates to treatments for improving cancer immunotherapy, and particular in solid tumors. The described treatments include sustained release of one or more chemotherapeutic agents, including oligonucleotides, including RNA interference agents.

Methods of treating cancer with the described treatments that additionally include administration of immunotherapy agents are also disclosed.

### BACKGROUND

Immunotherapies for cancer, also called Immuno-Oncology (TO), harnesses the immune system to treat cancer. IO agents use different methods, including checkpoint blockades, chimeric antigen receptor T cells, vaccination, and others. Checkpoint blockade, for example agents blocking the CTLA-4 and PD-1/PD-L1 checkpoints, have demonstrated a significant clinical response in some solid tumors. Many patients, however, do not respond to such therapies. In particular, patients with pancreatic cancer, the fourth most common cause of cancer-related deaths in the United States, showed no (or very limited) response in several clinical studies (Royal 2010, Brahmer 2012, Feig 2013, Javle 2016). The reasons for IO resistance are not completely understood, but apparently are associated with the limited infiltration of cytotoxic T-cell into solid tumors. Although immune cells are found to make up as high as 50% of pancreatic tumor cell mass, the immunosuppressive regulatory T (T reg) cells and myeloid derived suppressor cells (MDSCs) are predominant, with hardly any cytotoxic T lymphocytes (CTLs) infiltrating the tumors (Clark 2007). It has been proposed that there is a critical concentration below which the cytolytic activity of leukocytes, including CD8+ T cells is ineffective. Moreover, the vascular dysfunction in pancreatic tumors represents an additional major obstacle to systemic delivery of immunotherapy drugs (Feig 2012). Thus, a continuing need exists for improvements to immunotherapy approaches to cancer therapy, and in particular immunotherapy treatments for solid tumors. In particular, there is an urgent need to accelerate infiltration of T cells including CD8+ and CD4+ and of NK and NKT cells, into tumor cores.

### SUMMARY

Provided herein are compositions for use in conditioning a solid tumor in a subject to immunotherapy treatment. The described compositions include a polymeric drug delivery device comprising a chemotherapeutic agent, which upon delivery to the solid tumor and/or surrounding microenvironment, conditions the solid tumor to immunotherapy treatment.

Also provided herein are compositions for use in treatment of a solid tumor, which include a polymeric drug delivery device comprising a chemotherapeutic agent; and an immunotherapy composition.

It will be appreciated that the described compositions can all be used in the preparation of medicaments for use in conditioning a tumor and/or tumor microenvironment to immunotherapy and/or for use in treatment of a tumor.

Likewise, the described compositions can be used in methods for conditioning a tumor and/or tumor microenvironment for immunotherapy and/or in methods for treatment of a solid tumor.

The foregoing and other objects, features, and advantages will become more apparent from the following detailed description, which proceeds with reference to the accompanying figures.

### BRIEF DESCRIPTION OF THE DRAWINGS

FIGS. 1A-1C show the previously-published effects of siG12D-LODER on void volume in pancreatic tumors. Effects are shown microscopically (FIG. 1A) and are quantitated (bottom panels).

FIG. 1D is a bar graph showing the previously-published effects of siAR-LODER, siBMI1-LODER, and siHSP90-LODER on necrosis, and thus void volume, in a prostate tumor.

FIGS. 2A-2C show the effects of siG12D transfection on pancreatic tumor cell expression of TNF-alpha (FIG. 2A), IFN-beta (FIG. 2B), and IP-10 (FIG. 2C).

FIGS. 3A-3D show the effects of siG12D transfection on PBMC expression of IFN-alpha (FIG. 3A), IL-8 (FIG. 3B), IL1-RA (FIG. 3C), and MCP-1 (FIG. 3D) expression. Positive and negative controls for each condition are also shown.

FIGS. 4A-4B show that in-vivo, siG12D-LODER induces IFNβ in tumor tissue.

FIG. 4A: A 5 μg siG12D-LODER dose increases expression of IFNβ mainly in the tumor periphery. FIG. 4B. A 15 μg siG12D-LODER dose increases the expression of IFNβ across the entire tumor.

FIGS. 5A-5C show a comparison between a treated and an untreated sample for CD4 staining (identifying CD4+ T cells). Cells were counted using HALO™ of PerkinElmer/Indica Labs. FIG. 5A: Cells positive for CD4 staining were counted at varying distances from the LODER edge in the siG12D-LODER treated tumor. FIG. 5B: In the untreated tumor, CD4-positive cells were counted at varying distances from the tumor center. This was done by counting positively stained cells within 0.5 mm-wide concentric rings around the LODER edge/tumor center. All cells within each concentric ring were counted and the positively-stained cell concentration was calculated. The results show a higher concentration of CD4+ T cells in the middle of the tumor following siG12D-LODER treatment. FIG. 5C shows quantitative effect of siG12D-LODER on intratumoral T-cell concentration and distribution.

### BRIEF DESCRIPTION OF THE DESCRIBED SEQUENCES

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file named 2142 10 2 seq list_ST25, created May 23, 2018, about 6 KB, which is incorporated by reference herein. In the accompanying sequence listing:

SEQ ID NO: 1 is the sense strand of a siRNA targeting KRAS G12D.

SEQ ID NO: 2 is the anti-sense strand of a siRNA targeting KRAS G12D.

SEQ ID NO: 3 is the sense strand of a siRNA targeting androgen receptor (AR).

SEQ ID NO: 4 is the anti-sense strand of a siRNA targeting androgen receptor (AR).

SEQ ID NO: 5 is the sense strand of a siRNA targeting BMI1.

SEQ ID NO: 6 is the anti-sense strand of a siRNA targeting BMI1.

SEQ ID NO: 7 is the sense strand of a siRNA targeting HSP90.

SEQ ID NO: 8 is the anti-sense strand of a siRNA targeting HSP90.

SEQ ID NO: 9 is the anti-sense strand of a 2-O-met-modified siRNA targeting KRAS G12D.

SEQ ID NO: 10 is the sense strand of a a 2-O-met-modified siRNA targeting KRAS G12D.

SEQ ID NO: 11 is the cell penetrating peptide Tat.

SEQ ID NO: 12 is the cell penetrating peptide MPG.

SEQ ID NO: 13 is the cell penetrating peptide Pep-1.

SEQ ID NO: 14 is the sense strand of a siRNA targeting luciferase.

SEQ ID NO: 15 is the anti-sense strand of a siRNA targeting luciferase.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

Administration: The introduction of a composition into a subject by a chosen route. Administration of an active compound or composition can be by any route known to one of skill in the art. Administration can be local or systemic. Examples of local administration include, but are not limited to, topical administration, intratumoral administration, subcutaneous administration, intramuscular administration, intrathecal administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs (such as the drug delivery devices described herein), which release the active agents and compounds over extended time intervals for sustained treatment effects. An implantable device is “implanted” by any means known to the art of insertion into the tissue or tissue environment that is the area of a given treatment.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Altered expression: Expression of a biological molecule (for example, RNA (mRNA, miRNA, and the like) or protein) in a subject or biological sample from a subject that deviates from the expression if the same biological molecule in a subject or biological sample from a subject has normal or unaltered characteristics for the biological condition associated with the molecule. Normal expression can be found in a control, a standard for a population, and other similar baseline measures of expression. Altered expression of a biological molecule may be associated with a disease such as a cancer. The term associated with includes an increased risk of developing the disease as well as the disease itself. Expression may be altered in such a manner as to be increased or decreased. The directed alteration in expression of an RNA or protein may be associated with therapeutic benefits resulting from the direct effect on a molecule associated with a pathological condition, or from the indirect effect on such a molecule (e.g. wherein the altered expression results in changes in downstream expression that effect a pathology-related molecule).

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one light (about 25 kD) and one heavy chain (about 50-70 kD). The N-terminus of each chain defines a variable region of about 100 to 110 or more amino acids primarily responsible for antigen recognition. The terms variable light chain (V_{L}) and variable heavy chain (V_{H}) refer, respectively, to these light and heavy chains.

As used herein, the term antibody includes intact immunoglobulins as well as a number of well-characterized antibody fragments produced by digestion with various peptidases, or genetically engineered artificial antibodies. Thus, for example, pepsin digests an antibody below the disulfide linkages in the hinge region to produce F(ab)′₂, a dimer of Fab which itself is a light chain joined to V_{H}-C_{H} 1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially a Fab with part of the hinge region (see, Fundamental Immunology, W. E. Paul, ed., Raven Press, N.Y., 1993). While various antibody fragments are defined in terms of the digestion of an intact antibody, it will be appreciated that Fab′ fragments may be synthesized de novo either chemically or by utilizing recombinant DNA methodology. Thus, the term antibody as used herein also includes antibody fragments either produced by the modification of whole antibodies or synthesized de novo using recombinant DNA methodologies.

Antibodies for use in the methods, compositions, and systems of this disclosure can be monoclonal or polyclonal. Merely by way of example, monoclonal antibodies can be prepared from murine hybridomas according to the classical method of Kohler and Milstein (Nature 256:495-497, 1975) or derivative methods thereof. Detailed procedures for monoclonal antibody production are described in Harlow and Lane (Antibodies, A Laboratory Manual, CSHL, New York, 1988).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988). Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994). One or more CDRs may be incorporated into a molecule either covalently or noncovalently to make the resultant molecule an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

A neutralizing antibody or an inhibitory antibody is an antibody that inhibits at least one activity of a target—usually a polypeptide—such as by blocking the binding of the polypeptide to a ligand to which it normally binds, or by disrupting or otherwise interfering with a protein-protein interaction of the polypeptide with a second polypeptide. An activating antibody is an antibody that increases an activity of a polypeptide. Antibodies may function as mimics of a target protein activity, or as blockers of the target protein activity, with therapeutic effect derived therein.

Antisense inhibitor: Refers to an oligomeric compound that is at least partially complementary to the region of a target nucleic acid molecule to which it hybridizes. As used herein, an antisense inhibitor (also referred to as an “antisense compound”) that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression. Nonlimiting examples of antisense compounds include primers, probes, antisense oligonucleotides, antisense morpholinos, RNA interference (RNAi) agents, such as small (or short) interfering RNAs (siRNAs), micro RNAs (miRNAs), small (or short) hairpin RNAs (shRNAs), and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound.

Cancer: The product of neoplasia is a neoplasm (a tumor or cancer), which is an abnormal growth of tissue that results from excessive cell division. Neoplasia is one example of a proliferative disorder. A “cancer cell” is a cell that is neoplastic, for example a cell or cell line isolated from a tumor.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers (such as small cell lung carcinoma and non-small cell lung carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, menangioma, neuroblastoma and retinoblastoma). In particular embodiments, the cancer that is targeted for treatment by the described compositions and methods is a metastasis which is not the primary tumor.

Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Checkpoint inhibitory agent or checkpoint inhibitory antibody: an agent, particularly an antibody (or antibody-like molecule) capable of disrupting the signal cascade leading to T cell inhibition after T cell activation as part of what is known in the art the immune checkpoint mechanism. Non-limiting examples of a checkpoint inhibitory agent or checkpoint inhibitory antibody include antibodies to CTLA-4 (Uniprot P16410), PD-1 (Uniprot Q15116), PD-L1 (Uniprot Q9NZQ7), and B7H3 (CD276; Uniprot Q5ZPR3).

In the context of the present specification, a checkpoint agonist agent or checkpoint agonist antibody includes but is not limited to an antibody (or antibody-like molecule) capable of engaging the signal cascade leading to T cell activation as part of what is known in the art the immune checkpoint mechanism. Non-limiting examples of receptors known to stimulate T cell activation include CD122 and CD137 (4-1BB; Uniprot Q07011). The term checkpoint agonist agent or checkpoint agonist antibody encompasses agonist antibodies to CD137.

In certain embodiments, the immune checkpoint inhibitor agent is ipilimumab (Yervoy; CAS No. 477202-00-9).

In certain embodiments, the immune checkpoint inhibitor agent is an inhibitor of interaction of programmed cell death protein 1 (PD-1) with its receptor PD-L1. In certain embodiments, the immune checkpoint inhibitor agent is selected from the clinically available antibody drugs nivolumab (Bristol-Myers Squibb; CAS No 946414-94-4), pembrolizumab (Merck Inc.; CAS No. 1374853-91-4), pidilizumab (CAS No. 1036730-42-3), atezolizumab (Roche AG; CAS No. 1380723-44-3), and Avelumab (Merck KGaA; CAS No. 1537032-82-8).

Chemotherapeutic agent: An anti-cancer agent with therapeutic usefulness in the treatment of diseases characterized by abnormal cell growth or hyperplasia. Such diseases include cancer, autoimmune disease as well as diseases characterized by hyperplastic growth such as psoriasis. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Non-limiting examples of chemotherapeutic agents include ICL-inducing agents, such as melphalan (Alkeran™), cyclophosphamide (Cytoxan™), cisplatin (Platinol™) and busulfan (Busilvex™, Myleran™). Chemotherapeutic agents include small molecules, nucleic acid, peptide, and antibody-based therapeutic agents; examples of all of which are known in the art. Immunomodulatory agents, which enhance the activity of a subject's immune system against a foreign body, such as a tumor, including a solid tumor, are other examples of chemotherapeutic agents.

Drug Delivery Device (DDD): Device by which a therapeutic agent, such as an antisense inhibitor or chemotherapeutic agent, is provided to a subject. Non-limiting examples of DDDs include drug-eluting implants and stents. The LODER implant is described herein, in particular examples, for use with an RNAi agent, and is an illustrative DDD.

Effective amount of a compound: A quantity of compound sufficient to achieve a desired effect in a subject being treated. An effective amount of a compound can be administered in a single dose, or in several doses, for example daily, during a course of treatment. However, the effective amount of the compound will be dependent on the compound applied, the subject being treated, the severity, and type of the affliction, and the manner of administration of the compound.

Immunotherapy/Cancer Immunotherapy: Therapeutic treatment that modulates (activates or inhibits) immune system activity for treatment of a disease. As used herein, immunotherapy is used synonymously with cancer immunotherapy, which more particularly indicates that the target of an immunotherapy treatment is the targeting, inhibition, and/or elimination of a cancer cell and/or tumor. Non-limiting examples of immunotherapeutic agents include checkpoint inhibitors and activators, antibodies, natural and engineered immune cells, such as G-CSF lymphocytes, and T-cells engineered for adaptive cell transfer.

Injectable composition: A pharmaceutically acceptable fluid composition comprising at least one active ingredient, for example, a nucleic acid, including an RNAi agent, a peptide, or an antibody. The active ingredient is usually dissolved or suspended in a physiologically acceptable carrier, and the composition can additionally comprise minor amounts of one or more non-toxic auxiliary substances, such as emulsifying agents, preservatives, pH buffering agents and the like. Such injectable compositions that are useful for use with the compositions of this disclosure are conventional; appropriate formulations are well known in the art.

Increase sensitivity: Increase the sensitivity of a target cell, tissue, or organ to a given treatment. Increasing sensitivity can be measured, inter alia, by greater efficacy of the treatment, greater efficiency of the treatment, and the like. As described herein the sensitivity of a solid tumor to the effects of a cancer immunotherapeutic agent can be increased by prior or concurrent administration with a drug delivery device that delivers a chemotherapeutic agent to the tumor or surrounding tumor bed.

Local Drug EluteR (LODER): Millimeter scale drug delivery insertable device (DDD) or implant, composed of a polymer into which a given drug is incorporated. The drug, such as, but not limited to, an RNAi agent, small molecule, peptide, or antibody, is released into the surrounding environment over a period of time that will vary depending on the LODER composition. For example, in particular embodiments, LODER can release a drug over a period of hours, days, weeks, and even months. In addition to the polymer and a drug, LODER can contain agents which alter (modify) the hydrophobicity and/or pH associated with LODER manufacturing and/or internal environment in-vivo.

MicroRNA (miRNA): Short, single-stranded RNA molecule of typically 18-24 nucleotides long. Endogenously produced in cells from longer precursor molecules of transcribed non-coding DNA, miRNAs can inhibit translation, or can direct cleavage of target mRNAs through complementary or near-complementary hybridization to a target nucleic acid (Boyd, Lab Invest., 88:569-578, 2008). As used herein, a “microRNA sequence” includes both mature miRNA sequences and precursor sequences. As used herein, a microRNA “seed sequence” is a short sequence, generally about seven nucleotides long, that is fully complementary with the target nucleic acid.

Neoplasia, malignancy, cancer and tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Malignant tumors are also referred to as “cancer.”

Pharmaceutical agent: A chemical compound or composition capable of inducing a desired therapeutic or prophylactic effect when properly administered to a subject or a cell. Incubating includes exposing a target to an agent for a sufficient period of time for the agent to interact with a cell. Contacting includes incubating an agent in solid or in liquid form with a cell, such as contacting a tumor with the described siRNA in suspension or as incorporated into a drug delivery device.

Condition (a therapeutic target): Prepare or pre-treat a target tissue, organ, or cell, for an additional treatment, whereby the conditioning treatment improves the efficacy of the additional treatment. Efficacy improvement can be recognized in varied ways; non-limiting examples of which include a requirement of a smaller treatment dosage to achieve the same or improved effect, or efficacy with a standard dosage that is an improvement over treatment that does not follow conditioning. In particular embodiments, administration of a drug delivery device, such as a LODER, that provides a chemotherapeutic agent to a solid tumor (or surrounding tissue bed) can condition the tumor for immunotherapy treatment.

Preventing or treating a disease: Preventing a disease refers to inhibiting the development of a disease, for example inhibiting the development of myocardial infarction in a person who has coronary artery disease or inhibiting the progression or metastasis of a tumor in a subject with a neoplasm. Treatment refers to a therapeutic intervention that ameliorates a sign or symptom of a disease or pathological condition after it has begun to develop. In particular examples, treatment of a cancer can include inhibition of progression and/or prevention of a reoccurrence of the disease. In another example, treatment can include sensitizing or predisposing a tumor to an additional treatment, such as an immunomodulatory therapy.

Radiation Therapy (Radiotherapy): The treatment of disease (e.g., cancer or another hyperproliferative disease or condition) by exposure of a subject or their tissue to a radioactive substance. Radiation therapy is the medical use of ionizing radiation as part of cancer treatment to control malignant cells. Radiotherapy may be used for curative or adjuvant cancer treatment. It is used as palliative treatment where cure is not possible and the aim is for local disease control or symptomatic relief.

RNA interference (RNA silencing; RNAi): A gene-silencing mechanism whereby specific molecules, such as a double-stranded RNA (dsRNA), trigger the degradation of homologous mRNA (also called target RNA). Double-stranded RNA can be or is processed into small (or short) interfering RNAs (siRNA), which serve as a guide for cleavage of the homologous mRNA in the RNA-induced silencing complex (RISC). The remnants of the target RNA may then also act as siRNA; thus resulting in a cascade effect. An RNAi agent includes any nucleic acid that can either serve directly as siRNA, be processed into siRNA, or produce siRNA, for example DNA that is transcribed to produce RNA that in turn is processed into siRNA.

Sense/anti-sense strand: The strand of dsDNA containing the RNA transcript sequence (read from 5′ to 3′ direction) is the sense strand, and is also known as the “forward” strand. The opposite, reverse-complementary strand, which is used as the template for cellular RNA polymerase, is the antisense strand, and is also known as the “reverse” strand. Likewise, in a dsRNA molecule, the “sense” strand corresponds to the target gene coding sequence, and with the antisense strand, its reverse complement.

Small interfering RNAs: Synthetic or naturally-produced small double stranded RNAs (dsRNAs) that can induce gene-specific inhibition of expression in invertebrate and vertebrate species. These RNAs are suitable for interference or inhibition of expression of a target gene and comprise double stranded RNAs of about 15 to about 40 nucleotides containing a 3′ and/or 5′ overhang on each strand having a length of 0- to about 5-nucleotides, wherein the sequence of the double stranded RNAs is essentially identical to a portion of a coding region of the target gene for which interference or inhibition of expression is desired. The double stranded RNAs can be formed from complementary ssRNAs or from a single stranded RNA that forms a hairpin or from expression from a DNA vector.

Small molecule (inhibitor): A molecule, typically with a molecular weight less than 1000 Daltons, or in some embodiments, less than about 500 Daltons, which in particular embodiments, is capable of inhibiting, to some measurable extent, an activity of some target molecule.

Subject: Living multi-cellular organisms, including vertebrate organisms, a category that includes both human and non-human mammals.

Subject susceptible to a disease or condition: A subject capable of, prone to, or predisposed to developing a disease or condition. It is understood that a subject already having or showing symptoms of a disease or condition is considered “susceptible” since they have already developed it.

Target sequence: A target sequence is a portion of ssDNA, dsDNA, or RNA that, upon hybridization to a therapeutically effective oligonucleotide, results in the inhibition of expression of the target.

Therapeutically effective amount: A quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound.

Tumor bed: The tissue surrounding a solid tumor.

### II. Overview of Several Embodiments

Provided herein are compositions for use in conditioning a solid tumor in a subject to immunotherapy treatment. The described compositions include a polymeric drug delivery device comprising a chemotherapeutic agent, which upon delivery to the solid tumor and/or surrounding microenvironment, conditions the solid tumor to immunotherapy treatment.

In particular embodiments, the polymeric drug delivery device includes a biocompatible polymeric and or biodegradable polymeric matrix comprising a polymer selected from the group consisting of poly(glycolide-co-lactide) (PLGA), polylactic acid (PLA), polyglycolic acid (PGA), polyethylene glycol (PEG), and polycaprolactone (PCL).

In certain particular embodiments, the chemotherapeutic agent is a nucleic acid, peptide, small molecule, antibody or fragment thereof, or a combination thereof. For example, the nucleic acid can be a single-stranded RNA or a double stranded RNA interference (RNAi) agent, which in certain embodiments is the siRNA described herein as KRAS siG12D.

In other particular embodiments, wherein the polymeric drug delivery device (DDD) is a Local Drug EluteR (LODER) that includes 75-90% PLGA (85:15), 5-15% mannitol, and 0.1-0.5% sodium bicarbonate.

In yet further embodiments, the described compositions also include an immunotherapy composition, such as a PD-1 inhibitor, a PD-L1 inhibitor, or a CTLA-4 inhibitor. In particular exemplary embodiments, the PD-1 inhibitor is Pembrolizumab (Keytruda) or Nivolumab (Opdivo), or a biosimilar thereof; the PD-L1 inhibitor is Atezolizumab (Tecentriq), Avelumab (Bavencio), or Durvalumab (Imfinzi), or biosimilars thereof; and the CTLA-4 inhibitor is ipilimumab (Yervoy), or a biosimilar thereof.

Also provided herein are compositions for use in treatment of a solid tumor, which include a polymeric drug delivery device comprising a chemotherapeutic agent; and an immunotherapy composition.

In particular embodiments, the polymeric drug delivery device includes a biocompatible polymeric matrix comprising a polymer selected from the group consisting of poly(glycolide-co-lactide) (PLGA), polylactic acid (PLA), polyglycolic acid (PGA), polyethylene glycol (PEG), and polycaprolactone (PCL).

In certain particular embodiments, the chemotherapeutic agent is a nucleic acid, peptide, small molecule, antibody or fragment thereof, or a combination thereof. For example, the nucleic acid can be a single-stranded RNA or a double stranded RNA interference (RNAi) agent, which in certain embodiments is the siRNA described herein as KRAS siG12D.

In other particular embodiments, wherein the polymeric drug delivery device (DDD) is a Local Drug EluteR (LODER) that includes 75-90% PLGA (85:15), 5-15% mannitol, and 0.1-0.5% sodium bicarbonate.

In particular embodiments, the described compositions are for use in treating a solid tumor that is a pancreatic tumor.

In yet further embodiments, the described compositions also include an immunotherapy composition, such as a PD-1 inhibitor, a PD-L1 inhibitor, or a CTLA-4 inhibitor. In particular exemplary embodiments, the PD-1 inhibitor is Pembrolizumab (Keytruda) or Nivolumab (Opdivo), or a biosimilar thereof; the PD-L1 inhibitor is Atezolizumab (Tecentriq), Avelumab (Bavencio), or Durvalumab (Imfinzi), or biosimilars thereof; and the CTLA-4 inhibitor is ipilimumab (Yervoy), or a biosimilar thereof.

Uses of the described compositions for preparation of medicaments for the indicated treatments, and methods of treatment utilizing the described compositions as indicated by administration to a subject in need thereof are also described.

### III. Drug Delivery Devices for Enhancing Cancer Immunotherapy

Described herein is the observation that a chemotherapeutic agent that is delivered to a solid tumor by way of a polymeric delivery device, including a biodegradable polymeric delivery device, will induce physical changes in the tumor and specifically in the tumor microenvironment. Such changes described herein also are termed as “conditioning” the tumor and/or tumor microenvironment. These changes include but are not limited to changes in interstitial fluid pressure (IFP) and in ‘void volume’ (volume not occupied by cells) as for example described in Shemi et al, 2015, changes of cytokine and chemokine levels, and biological response including neoantigen presentation. Such changes in the tumor microenvironment are described herein as opening the tumor and tumor microenvironment to infiltration by specific and non-specific immune system agents (e.g. CD8 T-cells, NKT cells, cytokines, and the like). These observations indicate that such delivery of a chemotherapeutic agent by such compositions will condition a solid tumor to immunotherapy treatment.

In view of these observations, described herein are methods and compositions for use in conditioning a solid tumor to immunotherapy treatment. In the described methods, a biodegradable drug delivery device (DDD, or LODER) which includes a chemotherapeutic agent is implanted into a solid tumor or the surrounding tumor bed such that its chemotherapeutic payload is released to the tumor or surrounding area.

The DDD is generally composed of a biodegradable polymeric matrix; and at least one chemotherapeutic agent, such as an RNAi agent, wherein the chemotherapeutic agent is incorporated within the biodegradable polymeric matrix.

The described DDD can be a cylinder, a sphere, or any other shape suitable for an implant (i.e. that can be implanted in a subject). In particular embodiments, the DDD is of “millimeter-scale.” That is, a device whose smallest diameter is a least 0.3 mm. In certain embodiments, each of the dimensions (diameter, in the case of a sphere or cylinder; and height and/or width or length, in the case of a cylinder, box-like structure, cube, or other shape with flat walls) is between 0.3-10 mm, inclusive. In other embodiments, each dimension is between 0.5-8 mm, inclusive. In still other embodiments, each dimension is between 0.8-5.2 mm, inclusive, between 1-4 mm, inclusive, between 1-3.5 mm, inclusive, between 1-3 mm, inclusive, or between 1-2.5 mm, inclusive.

In particular embodiments, the device is a cylinder, having a diameter of 0.8 mm. In other preferred embodiments, the cylinder has a length of 5.5 mm. In other embodiments, the cylinder has a diameter of about 0.8 mm and a length of 5.5 mm. In other embodiments, a DDD of the described methods and compositions has the diameter of an 18-gauge needle. In other embodiments, the volume of the device is between 0.1 mm³ and 1000 mm³, between 0.2 mm³ and 500 mm³, between 0.5 mm³ and 300 mm³, between 0.8 mm³ and 250 mm³, between 1 mm³ and 200 mm³, between 2 mm³ and 150 mm³, between 3 mm³ and 100 mm³, or between 5 mm³ and 50 mm³.

In a particular embodiment, the DDD has a diameter of 0.8 mm and a length of 5.5 mm, containing 25% w/w siRNA, namely about 650 μg of siRNA.

In other embodiments, the w/w agent:polymer load ratio is above 1:100. In more preferred embodiments, the load is above 1:20. In more preferred embodiments, the load is above 1:9. In still more preferred embodiments, the load is above 1:3.

The DDD is composed of polymers, wherein the chemotherapeutic agent, such as a siRNA, release mechanism includes both bulk erosion of the polymer and diffusion of the chemotherapeutic agent; or in some embodiments, non-degradable, or slowly degraded polymers are used, wherein the main release mechanism is diffusion and the DDD includes surface erosion and/or bulk erosion, and in some embodiments the outer part of the DDD functions as membrane, and its internal part functions as a drug reservoir, which practically is separated and not affected by the surroundings for an extended period (for example from about a week to about a few months). Combinations of different polymers with or without several excipients, with different release mechanisms may also optionally be used. The concentration gradient at the surface is preferably constant during a significant period of the total drug releasing period, and therefore the diffusion rate is effectively constant (termed “zero mode” diffusion). The term “constant” refers to a diffusion rate that is maintained above the lower threshold of therapeutic effectiveness, but which may still optionally feature an initial burst and/or fluctuate, for example increasing and decreasing to a certain degree. In other embodiments, there is an initial burst of less than 10% of the total amount of drug, which may be considered negligible. In other embodiments, there is an initial burst of about 20% of the total amount of drug. In other embodiments, the design enables an initial strong burst of 30% or more of the total amount of drug. The diffusion rate is preferably so maintained for a prolonged period, and it can be considered constant to a certain level to optimize the therapeutically effective period, for example the effective silencing period.

In particular embodiments, the DDD releases a chemotherapeutic agent, such as an RNAi, agent in a controlled fashion, which will vary depending on factors including but not limited to the DDD's constituent polymers, additives, and surface-to-volume ratio. For example, decreasing the surface-to-volume ratio will increase the duration of RNAi agent release time.

The DDDs described herein are designed with a particular drug-release profile. One relevant parameter is the time point at which 95% of the active agent has been released. In some embodiments, the DDD releases 95% of the active agent in vivo, for example in a human prostate or in a pancreatic tumor, over a time period between 3-24 months inclusive, for example 3, 4, 5, 6, 8, 10, 12, 14, 16, 18, 20, 22, or 24 months and any duration in between, for example 3-12, 2-24, 2-15, or 3-10 months inclusive. Another relevant parameter is the time point at which 90% of the active agent has been released; this may be any of the aforementioned time frames.

Another relevant parameter is the percent of chemotherapeutic agent released at a given time point. For example, in some embodiments such as those in which the DDD is releasing an RNAi agent, 80-99% inclusive of the RNAi agent is released 3-months after implantation. In other embodiments, 80-99% of the active agent is released 2, 4, 6, 9, 12, or 24-months after implantation. Alternatively or in addition, in some embodiments no more than 30-50% of the RNAi agent is released from the DDD during the first 3 weeks after implantation. In certain embodiments, less than 5% of the RNAi agent is released from the DDD over a time period of 1 month starting from implantation. In other embodiments, less than 10% of the RNAi agent is released from the DDD over a time period of 1 month starting from implantation.

Delayed-release DDDs are utilized with the described chemotherapeutic agents. “Delayed-release”, as used herein, refers to DDDs that do not release more than 10% of the agent within the first 2 months (discounting an initial burst of up to 20%, which sometimes occurs). In other embodiments, the DDD does not release more than 10% of its drug load within the first 3 months. In particular embodiments, DDDs containing 1% trehalose exhibit delayed release.

In other embodiments, the DDD is coated (by dipping, spraying, or any other method known to those skilled in the art) with a slowly-degraded polymer that contains no drug. Various embodiments of slowly-degraded polymers are described herein, each of which can be utilized to create a delayed-release DDD. In some embodiments, the coating comprises a linear-chain monosaccharide; a disaccharide; a cyclic monosaccharide, a cyclic disaccharide. In other embodiments, the coating comprising an additive selected from lactose, sucrose, dextran, and hydroxyethyl starch. In yet other embodiments, the coating comprises mannitol. Alternatively, the coating may comprise trehalose. In still other embodiments, the coating does not comprise a sugar.

The DDD contains a biodegradable polymeric matrix into which the chemotherapeutic (e.g. RNAi) agent is incorporated. In particular embodiments, the matrix is composed of poly(lactic acid) (PLA). In other embodiments, the biodegradable matrix is composed of poly(glycolic acid) (PGA). In still other embodiments, the biodegradable matrix comprises the co-polymer of PLA and PGA known as poly(lactic-co-glycolic acid) (PLGA).

PLGA matrices of varying ratios of PLA:PGA are well known and are commercially available. Likewise, methods for making such matrices that incorporate RNAi agents are well known in the art. Exemplary methods are described in US Patent Application Pub. No. 2011/0195123. In particular embodiments, the PLA:PGA ratio in the PLGA copolymer is between 95:5 and 5:95, and more particularly between 25:75 and 75:25. In other embodiments, the ratio is between 50:50 and 75:25, meaning that the amount of co-polymer in the DDD includes between 50-75% PLA and between 25-50% PGA. In other embodiments, the PLA:PGA ratio is between 25:75 and 50:50, between 35:65 and 75:25, between 45:55 and 75:25, between 55:45 and 75:25, between 65:35 and 75:25, between 75:25 and 35:65, between 75:25 and 45:55, between 75:25 and 55:45, or between 75:25 and 65:25. In other embodiments, the PLA:PGA ratio is between 80:20 and 90:10, inclusive. In other embodiments, the PLA/PGA ratio is larger than 75:25, between 75:25 and 85:15, or between 75:25 and 95:5. Alternatively, the ratio is smaller than 25:75, between 25:75 and 15:85, or between 25:75 and 5:95. In some embodiments, the co-polymer has a PLA:PGA ratio of between 80:20 and 90:10, inclusive, for example 80:20, 82:18, 84:16, 86:14, 88:12, or 90:10. In other embodiments, the co-polymer has a PLA:PGA ratio larger than 75:25, for example 76:24, 78:22, 80:20, 82:18, 84:16, 86:14, 88:12, 90:10, 92:8, 94:6, 96:4, or 98:2. In yet other embodiments, the co-polymer has a PLA:PGA ratio smaller than 25:75, inclusive, for example 24:76, 22:78, 20:80, 18:82, 16:84, or 14:86, 12:88, 10:90, 8:92, 6:94, 4:96, or 2:98.

In other embodiments the biodegradable polymeric matrix is composed of PEG (poly (ethylene glycol)), which can be the majority of the DDD or used in combination with any other polymer described herein.

Other polymers that can be used in the described DDDs include tri-block PLA-PCL-PLA, wherein PCL denotes poly-caprolactone; Poly(D,L-lactide) (DL-PLA), poly(D,L-glycolide); or poly(D,L-lactide-co-glycolide). Design of biodegradable controlled drug-delivery carriers containing PLA, PGA, PEG, and/or PCL to have a specified release profile are described inter alia in Makadia and Siegel, 2011.

In some embodiments, a polymer used in the described DDDs has a molecular weight (MW) of greater than 5 kilodaltons (kDa). In other embodiments, the MW is greater than 50 kDa. In other embodiments, the MW is greater than 7 kDa, 10 kDa, 15 kDa, 20 kDa, 30 kDa, 70 kDa, 100 kDa, 150 kDa, or greater than 200 kDa. In other embodiments, the MW is between 5-100 kDa, between 7-80 kDa, 10-60 kDa, 20-50 kDa, or between 25-50 kDa. In a particular example extended, slow release (approximately 6 months) can be achieved with a DDD containing PLGA co-polymer having a high PLA:PGA ratio, such as 90:10, and a MW (molecular weight) higher than 50 KDa. A similar effect can be achieved by use of PLA.

In other embodiments, the biodegradable matrix further comprises one or more additives for a variety of purposes including modulating hydrophilic-hydrophobic interactions; enabling dispersion of the drug, eliminating aggregation; preserving the drug in hot-temperature or cold-temperature storage conditions; and facilitating creation of cavities in the implant that affect drug diffusion from the matrix.

Hydrophilic-hydrophobic interactions may cause aggregation of the active substance in cases of hydrophilic active substances, such as siRNA, incorporated within a hydrophobic polymer, resulting in aggregation during production or subsequently when the device is implanted into the body of a subject and is subjected for example to hydrolysis. Non-limiting examples of such an additive to reduce such interactions are open monosaccharides, for example mannitol; disaccharides such as trehalose; sorbitol; and other cyclic monosaccharides such as glucose, fructose, galactose and disaccharides such as sucrose or any other cryoprotectant. These additives also in some embodiments function by forming hydrogen bonds with biological molecules as water molecules are displaced, enabling the biological material to retain its native physiological structure and function. The above additives, when chiral, can be in the form of the D-enantiomer, the L-enantiomer, or a racemic mixture. Additional, non-limiting examples of such additives are lactose, sucrose, dextran, and hydroxyethyl starch.

In particular embodiments, the DDD has between 1% and 15% mannitol, such as 1%, 1.5%, 2%, 2.5%, 5%, 7.5%, 10%, or 12.5%, and 15%, or any amount between.

In other particular embodiments, the DDD has less than 5% trehalose, for example in different embodiments 0.5%, 1%, 1.5%, 2%, 2.5%, 3%, 3.5%, 4%, or 4.5%, the effects of which on RNAi agent release can be readily tested.

In other embodiments, the biodegradable matrix comprises an additive for protecting an agent, such as an RNAi agent, against low pH after implantation. The microenvironment in the DDD implant interior tends to be acidic. When delivering an RNAi agent, pH should preferably be maintained above a threshold. For example, polymers including PLGA and oligoneucliotides including RNAi drugs might degrade at pH<3. Accordingly, in more specific embodiments such as when the DDD is providing an RNAi agent to the solid tumor or tumor bed, such a pH modulating (i.e. pH-changing) additive may be selected from bicarbonates and carbonates, for example sodium bicarbonate, sodium carbonate, and magnesium hydroxide. In particular examples, sodium bicarbonate is included at a concentration between 0.05% to about 5%, such as about 1%. In other examples, sodium bicarbonate (or other pH modulating agent) is included at less than 1%, including 0.9%, 0.8%, 0.7%, 0.6%, 0.4%, and 0.2% or even less. In still other examples, sodium bicarbonate (or other pH modulating agent) is included at 2%, 3%, 4%, 5%, or any increment in between 1% and 5%.

The described DDDs can contain at least 10 μg of an RNAi agent, such as a siRNA. In other embodiments, the amount is between 10-2000 μg siRNA per device, including between 300-1700 μg siRNA per device, between 300-1100 μg siRNA per device, or between 400-900 μg siRNA per device. In particular embodiments, in addition or as an alternative to the RNAi agent described herein, other therapeutic agents can be incorporated into and delivered by the described DDDs. Non-limiting examples of such agents include an additional RNAi agent targeting other cancer-associated genes; small molecule chemotherapeutic agents, and other biologic immunotherapeutic agents such as but not limited to immunomodulating cytokines and monoclonal antibodies.

It is appreciated that multiple DDDs can be implanted in a given treatment. The amount of the RNAi agent in all the DDD's administered as a batch (a single dose) can be at least 4 μg, for example at least 5 μg, at least 6 μg, at least 7 μg, at least 8 μg, at least 10 μg, at least 12 μg, or at least 15 μg. In still other embodiments, the amount of RNAi agent present per dose is between 2-10 μg, inclusive, for example 2, 3, 4, 5, 6, 7, 8, 9, or 10 μg.

In yet other embodiments, all the DDD's administered as a batch deliver a dose of 0.008-0.065 mg/kg/month, inclusive, for example 0.008 mg/kg/month, 0.01 mg/kg/month, 0.015 mg/kg/month, 0.02 mg/kg/month, 0.03 mg/kg/month, 0.05 mg/kg/month, or 0.065 mg/kg/month.

In certain embodiments, the drug percentage of the described DDDs is at least 20%. In another embodiment, the drug percentage is at least 30%, for example 30%, 35%, 40%, 45%, 50%, 55%, or 60%. In another embodiment, the drug percentage is between 8-30%, inclusive, for example 8%, 9%, 10%, 11%, 12%, 13%, 14%, 15%, 16%, 17%, 18%, 19%, 20%, 22%, 24%, 26%, 28%, or 30%.

As described, a large variety of DDDs can be contemplated, taking the various amounts of polymer, RNAi agent and optional additives. Particular non-limiting examples of such DDDs follow.

In a particular embodiment, the DDD (LODER) contains 64-76% PLGA (with a ratio of PLA:PGA at 90:10); 16-27% RNAi agent; and 5-12% mannitol, with or without 0.05%-1.5% sodium bicarbonate. In other particular embodiments the DDD can be 80-85% PLGA (with a ratio of PLA:PGA at 85:15); 10-12% siRNA; 7.5-10% mannitol, and 0.1-0.3% sodium bicarbonate;

In still other embodiments, the described DDD contains trehalose and not mannitol. In still other embodiments, the DDD comprises both trehalose and mannitol. In more specific embodiments, the DDD may contain 70-91.2% PLGA; 8-30% siRNA; 0.6-1.5% trehalose; and 0.1-0.4% sodium bicarbonate. In other embodiments, the DDD may contain 75-91.2% PLGA; 8-25% siRNA; 0.6-1.5% trehalose; and 0.1-0.4% sodium bicarbonate. In still other embodiments, the DDD may contain 80-91.2% PLGA; 8-20% siRNA; 0.6-1.5% trehalose; and 0.1-0.4% sodium bicarbonate. In yet other embodiments, the DDD may contain 85-91.2% PLGA; 8-15% siRNA; 0.6-1.5% trehalose; and 0.1-0.4% sodium bicarbonate. In additional embodiments, the DDD may contain 88-91.2% PLGA; 8-12% siRNA; 0.6-1.5% trehalose; and 0.1-0.4% sodium bicarbonate. In yet other embodiments, the DDD may contain 89-91% PLGA; 8-10% siRNA; 0.6-1.5% trehalose; and 0.1-0.4% sodium bicarbonate. In still other embodiments, the DDD may contain about 90% PLGA 85:15, about 9% siG12D, about 1% Trehalose, and about 0.2% NaHCO₃. In any of the above DDD formulations, the siRNA can be replaced by an alternative chemotherapeutic agent, such as an alternative nucleic acid, small molecule, or peptide-based agent (e.g. an antibody).

In other embodiments, the described DDDs can be coated. A coating can be designed for a number of characteristics, including modulating the release rate or preventing protein stickiness during long-term storage. The coating in some embodiments comprises the same material used to form the matrix, for example a PLGA co-polymer matrix with or without the additives or with additives with different ratio but without a chemotherapeutic agent (e.g. RNAi agent). In other embodiments, the coating comprises a material similar to that used to form the matrix (for example containing the same building blocks in a different ratio, or containing the same polymer but with a different MW), only without the RNAi agent. In other embodiments, the coating comprises the same material used to form the matrix, together with at least one other polymeric material such as PEG. In other embodiments, the coating includes PLA. In still other embodiments, the coating includes a PLGA co-polymer wherein the PLA:PGA are in a ratio of at least 80:20, for example 80:20, 82:18, 84:16, 85:15, 86:14, 88:12, 90:10, 92:8, 94:6, 96:4, 98:2, and 99:1, and having a MW greater than 50 KDa, for example 60 KDa, 70 KDa, 80 KDa, 100 KDa, 120 KDa, 1500 KDa, or 200 KDa).

In particular embodiments, the described DDDs also contain chemotherapeutic agent-complexed small particles, which are distributed within the biodegradable polymeric matrix of the DDD. Small particles include “microparticles” and “nanoparticles,” Microparticles include particles having a size within the range 800 nm-5 μm (also referred to as microspheres). Nanoparticles include particles of size within the range 4 nm-800 nm. (The lower size of ˜4 nm typifies the smaller particles described here, which in typical embodiments is not a sphere, but a molecular complex, for example a drug molecule such as a siRNA molecule that is complexed with a polymer or conjugated to an additional molecule(s)).

In certain embodiments, the particles comprise a polymeric material as described herein, which can be different from or identical to that in the matrix.

“Different from” refers to a polymer made from different building blocks from that in the matrix or even sharing at least one building block with the polymer in the matrix, but having a different composition. For example, the particles can be composed of PLA, whereas the surrounding matrix of the DDD can be composed of PLGA. In another example the differences between the polymers in the particles and the DDD matrix include polymers containing a particular enantiomer as opposed to a racemic mixture of a given building block (L-PLA vs. DL-PLA), polymers containing the same building blocks in a different ratio (having either the same or different molecular weight (MW)), or containing the same building blocks but having a different MW (having either the same or different ratio). “Identical to” refers to polymers with the same building blocks, in the same ratio, and with the same MW.

It will be appreciated that particles composed of polymers that are “identical to” the constituent polymer of the DDD matrix can contain additional materials that are different from the matrix. In particular embodiments, the polymer in the particles is non-identical to the polymer in the matrix.

In still other embodiments, the small particles do not comprise a polymeric-matrix. For example, the particles may be liposomes. Other examples include particles comprising DOTAP or PEI, or another cationic molecule complexed with the RNAi agent, as similarly described above.

In particular embodiments of DDDs that include agent-complexed small particles, particle complexes, for example siRNA-DOTAP complexes, are dissolved in chloroform and incorporated within larger PLA particles. Such particles are then suspended in ethyl acetate and mixed with PLGA to form a matrix.

In particular examples both the DDD matrix and the small particles are complexed with a RNAi agent. In other examples, the DDD matrix is not complexed with the RNAi agent, but the suspended particles are complexed with the RNAi agent. In those embodiments wherein both the DDD matrix and the particles are complexed with the RNAi agent, the RNAi agent can be the same in the matrix and particles or different in the matrix and particles.

Additional examples of DDDs containing small particles, including constituent components, methods of production and the like, can be found in US Patent Publication No. 2013/0122096, the contents of which are incorporated by reference herein in their entirety.

In particular embodiments, an RNAi agent for use in the described methods and compositions is a short (or small) interfering RNA (siRNA), short hairpin RNA (shRNA), or microRNA. In other embodiments RNAi agents include longer polynucleotide molecules that are processed intracellularly to yield siRNA. Particular examples include DsiRNA, which are cleaved by the RNase III class endoribonuclease dicer into 21-23 base duplexes having a 2-base 3′-overhang; UsiRNAs, which are duplex siRNAs that are modified with non-nucleotide acyclic monomers, termed unlocked nucleobase analogs (UNA), in which the bond between two adjacent carbon atoms of ribose is removed, and which may be designed to enter the RNAi pathway via Dicer enzyme or directly into RISC; self-delivering RNA (sdRNA) such as rxRNA® of RXi Therapeutics, and agents inhibiting the pre-mRNA maturation step of polyA tail addition such as the U1 adaptor (Integrated DNA Technologies (IDT) Inc).

In certain embodiments, the RNAi agent is between 25-30 nucleotides (nt) in length, such as 25-27 nt and 19-25-nt. In other embodiments, the RNAi agent is 19 nt long. In other embodiments, the sense strand and/or the antisense strand further comprises a 1-6-nt 3′-overhang. In particular embodiments, the RNAi agent is 100% complementary to its target sequence. In other embodiments, the RNAi agent is only partially complementary, with 1, 2, 3 or more nucleotides that are different from its target sequence. In other embodiments, a two-base 3′ overhang is present. In more specific embodiments, the sense strand and the antisense strand each further comprises a 2-nt 3′-overhang. In still further embodiments, the 3′ overhangs are made from consecutive deoxythymine (dT) nucleotides, such that a 2 nt 3′ overhang is dTdT. In other embodiments, a siRNA used in the described methods and compositions has a 19+2 overhang design, namely sense and anti-sense of 19 base-paired nucleotides and two unpaired nucleotides at the 3′ end of each of the strands. In certain embodiments, as exemplified herein, the overhangs are each dTdT.

In other embodiments, one or more nucleotides of the described RNAi agent are modified by 2′-OMe or 2′-F. In particular embodiments, such modifications are made in one or both strands of a described siRNA. The described modified sequences may be used with or, in other embodiments without, overhangs at the 3′ end of each of the strands (in the instance of a dsRNA RNAi agent). In certain embodiments, the overhangs each consist of two unpaired nucleotides. In more specific embodiments, as exemplified herein, the overhangs are each dTdT (2 deoxythymidine residues).

In other embodiments, the described RNAi agents can be chemically modified, separate from or in addition to the modifications described above. In a particular embodiment, the modification is a backbone or linkage modification. In another embodiment, the modification is a nucleoside base modification. In a further embodiment, the modification is a sugar modification. In more specific embodiments, the modification, including the nucleotide modifications described above, is selected from the modifications appearing in Table 1 below. In other embodiments, the modification is selected from a locked nucleic acid (LNA) and/or peptide nucleic acid (PNA) backbone. Other modifications are described in US Patent Application Pub. No. 2011/0195123.

TABLE 1
RNAi agent modifications

| | |
|---|---|
| Modification | Position of the substitution |

Sugar modifications

| | |
|---|---|
| dNTPs- dTdT | 3′-overhangs of sense and/or anti-sense strands |
| dNTPs- dNPs | Any number of residues in the sense strand; 0-4 |
| | residues at the 5′ end of the antisense strand |
| 2′-O-methyl (2′OMe) | Any number of residues in the sense and/or |
| rNPs | antisense strands |
| 2′-fluoro (2′-F) rNPs | Any number of pyrimidine residues in the sense |
| | and/or antisense strands |
| combined use of | Any number of pyrimidine residues in the sense |
| 2′OMe and 2′-F | and/or antisense strands to 2′-F; and any number |
| | of purine residues in the sense and antisense |
| | strands to 2′-OMe. |
| 2′-O-(2-methoxyethyl) | Any number of pyrimidine residues in the sense |
| (MOE) rNPs | and/or antisense strands |
| 2′-fluoro-β-D (FANA) | Any number of pyrimidine residues in the sense |
| rNPs | strand |
| Locked nucleic acids | from none till 4 last ribonucleotides at the 3′ end |
| (LNA) | of the sense strand; and 3′ overhangs of the |
| | antisense strand |
| combined use of DNA | substitution of any number of pyrimidine (T and |
| and 2′-F | C) ribonucleotides to 2′-F ribonucleotides and |
| | any number of purines (A and G) to |
| | deoxyribonucleotides in sense and/or antisense |
| | strands |

phosphate linkage modifications - phosphorothioate (PS)

| | |
|---|---|
| phosphodiester | substitution of any number of ribonucleotides in |
| | sense and/or antisense strands |
| phosphorothioate (PS) | substitution of any number of ribonucleotides in |
| | sense and/or antisense strands |
| boranophosphate DNA | substitution of any number of ribonucleotides in |
| or RNA | sense and/or antisense strands |
| amide-linked | substitution of any number of ribonucleotides in |
| | sense and/or antisense strands |
| phosphoramidate | substitution of any number of ribonucleotides in |
| | sense and/or antisense strands |
| methylphosphonate | substitution of any number of ribonucleotides in |
| | sense and/or antisense strands |
| 2′,5′-linked | substitution of any number of ribonucleotides |
| DNA or RNA | in sense strand |

Base_modifications

| | |
|---|---|
| 5-bromouracil | substitution of any number of ribouracils in |
| (5-Br-Ura) | sense and/or antisense strands |
| 5-iodouracil (5-I-Ura) | substitution of any number of ribouracils in |
| | sense and/or antisense strands |
| dihydrouracil | substitution of any number of ribouracils in |
| | sense and/or antisense strands |
| 2-thiouracil | substitution of any number of ribouracils in |
| | sense and/or antisense strands |
| 4-thiouracil | substitution of any number of ribouracils in |
| | sense and/or antisense strands |
| pseudouracil | substitution of any number of ribouracils in |
| | sense and/or antisense strands |
| diaminopurine | substitution of any number of adenines in both |
| | sense and/or antisense strands |
| difluorotoluene | substitution of any number of adenines in both |
| | sense and/or antisense strands |
| peptide nucleic acids | substitution of any number of ribonucleotides in |
| (PNAs) | sense and/or antisense strands |
| (2-aminoethylglycine) | |

modifications to the overhangs and termini

| | |
|---|---|
| 2-nt-3′-DNA overhang | 3′ end of sense and/or antisense strands |
| 2-nt-3′-RNA overhang | 3′ end of sense and/or antisense strands |
| blunt-ended duplexes | 3′ end of sense and/or antisense strands |

chemical conjugation

| | |
|---|---|
| cholesterol | covalently attached to sense and/or antisense |
| | strands |
| vitamin-E | covalently attached to sense and/or antisense |
| (α-tocopherol) | strands |

In other embodiments, the described RNAi agent may be conjugated to cholesterol, a cell penetrating peptide, or alpha-tocopherol-vitamin E. In certain embodiments wherein the RNAi agent is double-stranded, the cholesterol may be conjugated to the 3′ end of the sense strand. In other embodiments, the cholesterol may be conjugated to the 5′ end of the sense strand. In certain embodiments, in the case of a hairpin-shaped molecule, the cholesterol may be conjugated to the loop. These and further examples of conjugating molecules are described in US Patent Application Pub. No. 2011/0195123.

In certain embodiments, the RNAi agent is associated, either via covalent attachment or via non-covalent complexation, with a cell-penetrating peptide (CPP), also referred to as protein transduction domains (PTDs), which can facilitate the delivery of a molecular cargo to the cytoplasm of a cell. Non-limiting examples of CPP's include HIV-1 Tat (NCBI Gene ID: 155871) or a fragment thereof comprising the sequence YGRKKRRQRRR (SEQ ID No: 11); pAntp (penetratin) (NCBI Gene ID: 40835); Is1-1 (NCBI Gene ID: 3670); Transportan, Pooga et al), MPG (GALFLGFLGAAGSTMGA [SEQ ID No: 12); and Pep-1 (KETWWETWWTEW; SEQ ID No: 13). CPP's are known to those skilled in the art and are described inter alia in Deshayes et al.

In other embodiments, the described RNAi agents may be complexed with a cationic molecule, such as DOTAP (N-[1-(2,3-Dioleoyloxy)propyl]-N,N,N-trimethylammonium), DOPE (1,2-dioleoyl-sn-glycero-3-phosphatidylethanolamine), 1,2-dioleoyl-sn-glycero-3-phosphocholine (DOPC), 1,2-di-O-octadecenyl-3-trimethylammonium propane (DOTMA), spermine, PEI (polyethylenimine), a PEI-PLA polymer, or N-Acetylgalactosamine (GalNAc).

In particular embodiments, the RNAi agents are formulated for systemic delivery, in other embodiments, the RNAi agents are formulated for local delivery to an area of treatment.

In still other embodiments, in the described methods and compositions utilize an alternative chemotherapeutic (also described herein as an “anti-cancer”) agent as an alternative or in addition to an RNAi agent.

In more specific embodiments, the chemotherapeutic agent includes a pyrimidine analogue, non-limiting examples of which are 5-azacytidine, 5-aza-2′-deoxycytidine, 5-fluoro-uracil, 5-fluoro-deoxyuridine (floxuridine), and 5-fluorodeoxyuridine monophosphate. In more specific embodiments, the anti-cancer agent is an inhibitor of ribonucleoside-diphosphatereductase large subunit (EC 1.17.4.1), non-limiting examples of which are motexafin gadolinium (CHEBI: 50161); hydroxyurea; gemcitabine (2′,2′-difluorodeoxycytidine); elacytarabine (CP-4055; an ara-C-5′elaidic-acid-ester) and CP-4126, (CO 1.01; a gemcitabine-5′elaidic-acid-ester; Adema A D et al, Metabolism and accumulation of the lipophilic deoxynucleoside analogs elacytarabine and CP-4126. Invest New Drugs. 2011 Oct. 15), and those described in WO2011062503, the contents of which are incorporated herein by reference. In even more specific embodiments, the anti-cancer agent comprises gemcitabine. In alternative embodiments, the anti-cancer agent is gemcitabine. In yet other embodiments, the anti-cancer agent is an EGFR tyrosine kinase inhibitor. In yet other embodiments, the anti-cancer agent comprises a thymidylate synthase inhibitor. In more specific embodiments, the anti-cancer agent comprises leucovorin (Folinic acid; 2-[[4-[(2-amino-5-formyl-4-oxo-1,6,7,8-tetrahydropteridin-6-yl)methylamino]benzoyl]amino]pentanedioic acid). In yet other embodiments, the anti-cancer agent comprises irinotecan. In yet other embodiments, the anti-cancer agent comprises oxaliplatin. In still other embodiments, the anti-cancer agent comprises FOLFIRIN (5-fluorouracil, leucovorin, and irinotecan in combination). In still other embodiments, the anti-cancer agent is FOLFIRINOX (5-fluorouracil, leucovorin, irinotecan, and oxaliplatin in combination), or any combination of a subset of the four agents in FOLFIRINOX. In one embodiment the agent administered in addition to a DDD can be Modified FOLFIRINOX, administrated as follows: Oxaliplatin (85 mg/m²)—IV for 2 hours, immediately followed by Irinotecan (180 mg/m²)—IV for 90 min. Leucovorin—400 mg/m², followed by a Fluorouracil continuous IV infusion of 2,400 mg/m² (over 46 hours) every two weeks. In yet other embodiments, the anti-cancer agent comprises an EGFR tyrosine kinase inhibitor. In more specific embodiments, the anti-cancer agent is Erlotinib. In particular embodiments, anti-cancer agent is administered in addition to the DDD and is administered concurrently, prior to, or following administration of an anti-cancer agent.

The methods and compositions described herein are used for conditioning a cancer (e.g. a solid tumor) for immunotherapy. In particular embodiments, the cancer is a prostate carcinoma. In other nonlimiting embodiments, the cancer is another cancer such as a cancer selected from a pancreatic tumor, a colon tumor, a lung tumor, brain cancer, liver cancer, kidney cancer, melanoma, endometrial carcinoma, gastric carcinoma, renal carcinoma, biliary carcinoma, cervical carcinoma, and bladder carcinoma. In more specific embodiments, the cancer is selected from pancreatic carcinoma, pancreatic ductal adenocarcinoma, small-cell lung carcinoma, and colorectal cancer.

In particular embodiments, a mixture of delayed release and non-delayed release DDDs are implanted into the subject. Provision of a combination of delayed-release and non-delayed-release DDD's in some embodiments enables a longer time course of significant chemotherapeutic agent (e.g. siRNA) release, without the need for repeated therapeutic intervention.

In some embodiments, the described DDD is implanted intratumorally. In other embodiments, the DDD is implanted into the vicinity of the tumor. In more specific embodiments, in the case of a well-defined solid tumor, several devices are spaced within the tumor volume. In yet other embodiments, several devices are implanted along a needle cavity within the tumor. In still other embodiments, the device or devices are implanted such that they are not in a direct contact with the perimeter of the tumor. Alternatively, in the case of a poorly defined solid tumor, the device is inserted into an area believed to contain tumor cells.

As shown herein, use of a chemotherapeutic agent-delivering DDD will condition a solid tumor to immunotherapy treatment. Accordingly, concurrent with, or following implantation of the DDD, an immunotherapy is provided to the subject.

In particular embodiments, the immunotherapy composition includes an immune checkpoint inhibitor such as a PD-1, PD-L1, and CTLA4 inhibitor. Non-limiting examples of immunotherapy agents for use in the described methods and with the described composition include Nivolumab (Opdivo®), a PD-1 antibody; Ipilimumab (Yervoy®), a CTLA-4 antibody; Pembrolizumab (Keytruda®, MK-3475), a PD-1 antibody; antibodies developed by CStone Pharmaceuticals: CS1001 (anti-PD-L1), CS1002 (anti-CTLA-4), and CS1003 (anti-PD-1); ZKAB001 (anti-PD-L1, China Oncology Focus/Sorrento Therapeutics); SHR-1210 (anti-PD-1, Shanghai Hengrui Pharmaceutical Co. Ltd.); JS-001 (anti-PD-1, Shanghai Junshi Biosciences Co., Ltd.); IBI308 (anti-PD-1, Innovent Biologics); PLX3397, a tyrosine kinase inhibitor of KIT, CSF1R, and FLT3; MGA271, an antibody that targets B7-H3; Reolysin®, an oncolytic virus that is able to replicate specifically in cancer cells bearing an activated RAS pathway; Durvalumab (MEDI4736): a PD-L1 antibody+/−Tremelimumab, a CTLA-4 antibody; MGD009, a B7-H3×CD3 DART protein; R070097890, a CD40 antibody; algenpantucel-L; NY-ESO-1 protein in patients with advanced cancer whose cancers express NY-ESO-1; GVAX vaccine+/−nivolumab; dendritic cell vaccine; TERT vaccine; IL-12 vaccine; Adaptive cell therapies, e.g. T-cells engineered to target specific antigens; Monoclonal antibody therapies (in addition to those listed above); Adjuvant immunotherapies; and Cytokine therapy.

The above and other immunotherapeutic agents are listed online at cancerresearch.org/cancer-immunotherapy/impacting-all-cancers/pancreatic-cancer. Further immunotherapeutic agents include for example, MPDL3280A, an anti-PD-L1 antibody, PF-05082566, an anti-4-1BB/CD137 antibody, and Urelumab (BMS-663513). Other immunotherapeutic agents include SIRP-alpha antagonist: OSE-172 (Boehringer Ingelheim and OSE Immunotherapeutics) CD47 antagonists: Hu5F9-G4—(Forty Seven), CC-90002 (Celgene), TTI-621 and TTI-622 (Trillium Therapeutics). STING (Stimulator of Interferon Genes) activators: ADU-S100/MIW815 (Aduro Biotech), and MK-1454 (Merck). Similarly, immunotherapy agents are listed in Liu and Wu, 2017.

The therapeutic methods described above can be associated with or without additional administration of any of the chemotherapeutic agents described above. For example, Gemcitabine alone, Gemcitabine+Abraxane, Erlotinib, the FOLFIRINOX drug combination (fluorouracil [5-FU], leucovorin, irinotecan and oxaliplatin) or one or a few of this combination.

In particular embodiments, the drug delivery device is inserted directly into a tumor, or into the surrounding tumor bed, and the immunotherapy agent is administrated systemically or locally. In other embodiments, the immunotherapy agent is included in the DDD, and is accordingly directly delivered into, or in the area of, the tumor.

In other embodiments, the immunotherapy agent is administered to the patient separately from the polymeric drug delivery device, whether by separate local injection or by systemic methods of administration (IV, IP, intramuscular, or even oral methods).

In particular embodiments, the polymeric drug delivery device is inserted into the tumor/tumor bed in a single administration, followed by multiple administrations of the immunotherapy agent over the course of 1, 2, 3, 4, 5, 6 weeks or 1, 2, 3, 4, 5, 6, months or up to 36 months or longer.

In still further embodiments, the treatments described herein can include traditional/standard-of-care chemotherapeutic agents administered systemically in addition to, or in place of, the immunotherapy agents as described.

In still other embodiments, the described methods include administering radiation therapy to the patient. In some embodiments, the radiation is administered to the patient after administration of the DDD, such as up to 10 days after administration of the DDD. Alternatively, the radiation is administered to the patient simultaneously with administration of the DDD. In still other embodiments, the radiation is administered to the patient before administration of the DDD. In yet other embodiments, the DDD is administered during ongoing administration of the radiation.

The following examples are provided to illustrate certain particular features and/or embodiments. These examples should not be construed to limit the disclosure to the particular features or embodiments described.

### EXAMPLES

### Example 1: siRNA-Delivering LODER Increases Tumor Necrosis and Void Volume

The siG12D-LODER is a millimeter-scale polymeric agent that contains a siRNA targeted against mutated KRAS. It is inserted (not injected) by ultrasound guided endoscopy into a tumor and releases the siRNA continuously for at least four months. Mutated KRAS dominates more than 90% of the pancreatic cancer patient population, but so far KRAS has been considered an ‘undruggable’ target using classical small molecule approaches. siG12D-LODER has been studied in pre-clinical animal models, standard animal toxicology protocols, and in Phase 1/2a clinical studies (Zorde-Khvalevsky 2013 and Shemi 2015; Ramot 2016; Golan 2015, respectively). In preclinical models, we have observed a correlation between specific silencing of KRAS-G12D with cellular apoptosis and necrosis. These effects persist for months.

An additional effect of the siG12D-LODER on the tumor microenvironment, a change in tumor void volume (volume not occupied by cells), was studied in the experiment described here.

Tumor xenografts were established by subcutaneous injection of 10⁶ log-phase growth viable Panc 02 cells in 100 μL PBS into female C57BL/6 mice. The cells were injected into the flanks of the mice. When tumors reached an average volume of 80 mm³, LODERs containing 5 μg siG12D, siLuc or empty LODERs were implanted into tumors under anesthesia.

The LODER (also described herein as the DDD) is a smooth, miniature, rod-shaped polymeric (PLGA matrix) tube embedded with the drug substance. The siG12D-LODERs used for implantation in mice were with a length of ˜2.5-3 mm and a diameter of ˜0.9 mm. Several LODER formulations, as described herein, have been tested with similar effect. The experiments presented in the current and following examples employed LODERs that include about 80-85% PLGA (85:15); about 10-12% siRNA, as indicated; about 5-10%% mannitol, and about 0.1-1% sodium bicarbonate.

The following sequences were used in the experiments described herein. In the following sequences, uppercase letters denote RNA bases and underlined bold uppercase letters denote DNA bases. All inter-nucleotide linkages are phosphodiester:

siG12D Sequence:
Sense:
(SEQ ID NO: 1)
5′-GUU GGA GCU GAU GGC GUA GTT-3′
Antisense:
(SEQ ID NO: 2)
5′-CUA CGC CAU CAG CUC CAA CTT-3′
siLuc Sequence:
Sense:
(SEQ ID NO: 14)
5′-CUU ACG CUG AGU ACU UCG ATT
Antisense:
(SEQ ID NO: 15)
5′-UCG AAG UAC UCA GCG UAA GTT

At different time points after treatment, mice were killed and tumors were formalin fixed and paraffin embedded (FFPE). For histological analysis, tissues were cut to 5 μm sections, which were stained with Hematoxylin and eosin (H&E). H&E staining allows detection of cellular and tissue structures.

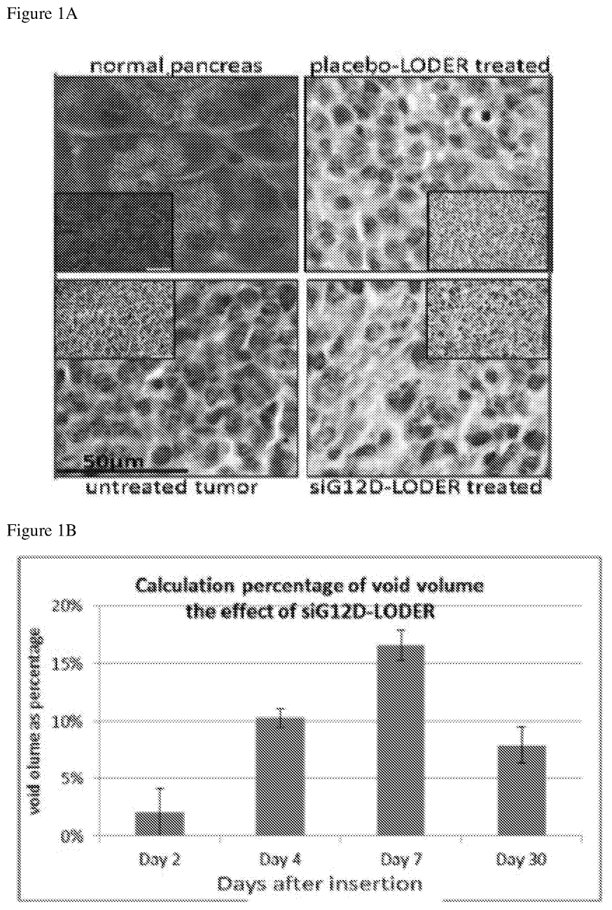

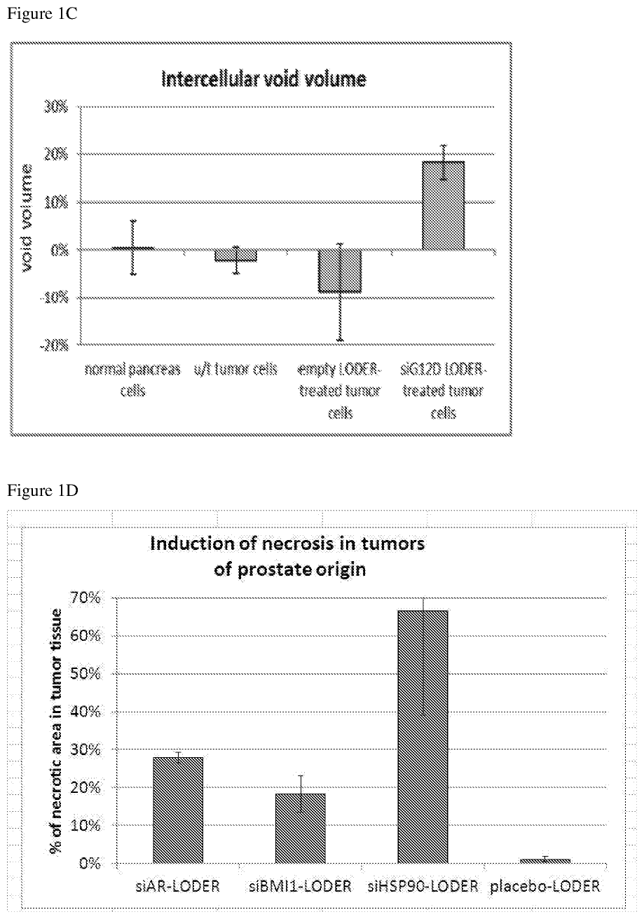

About a week after insertion of the siG12D-LODER, we observed changes in the tumor microenvironment, with the ‘void volume’ increasing from ˜0% to ˜10% of the field (see FIGS. 1A-1C). This increase in void volume represents an opening of the local tumor environment (Shemi et al 2015).

The necrotic effect of a siRNA-delivering LODER is not a cell-type or siRNA specific-effect. As shown in FIG. 1D (adapted from US 2017/0283803), LODER-delivering siRNA for targeting the Androgen Receptor gene (siAR-LODER; SEQ ID NOs 3 and 4), the BMI1 gene (siBMI1-LODER; SEQ ID NOs 5 and 6), and the Heat Shock Protein 90 gene (siHSP90-LODER; SEQ ID NOs 7 and 8) also induces increased tumor necrosis.

### Example 2: Non-Specific Immunostimulatory Effects of siRNA Transfection and siRNA-LODER

In addition to specific gene silencing and/or translation arrest drugs, oligonucleotides, including antisense, micro-RNA, dsRNA and siRNA, can also trigger nonspecific immune stimulation. Indeed, in pre-clinical studies, siG12D transfection (in vitro) and siG12D-LODER (in vivo) raised levels of tumor suppressing cytokines including IFNα, IFNβ and TNF.

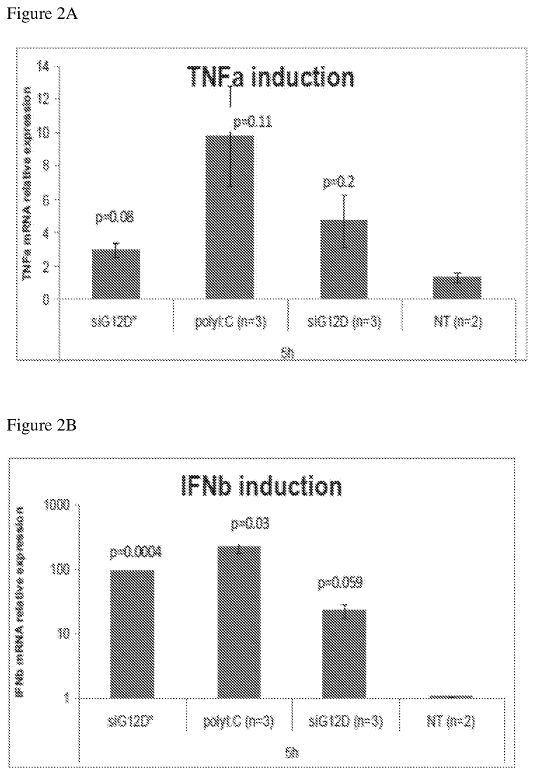

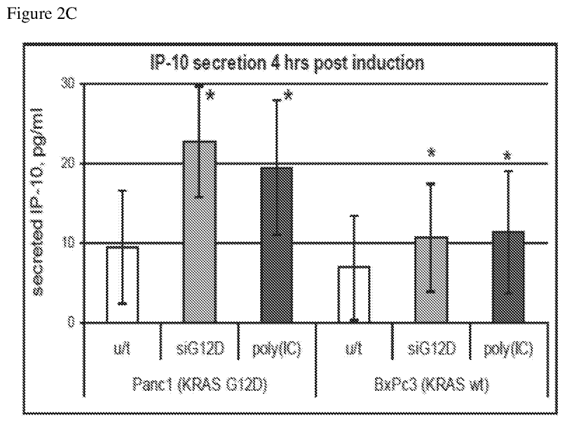

In order to study the potential of siG12D to induce an innate immune response, PANC-1 cells were either transfected or incubated with siG12D or siG12D-8′ (2-O-met-modified siRNA, SEQ ID NOs 9 and 10, was used for comparison). Incubation with poly (I:C) was used as a positive control. TNF-α and IFN-β induction was assessed by Real-Time PCR, using commercially available PCR primers (FIG. 2A-B).

These results show that PANC-1 cells transfected with siG12D, siG12D will induce TNF-α and IFN-β expression.

To determine whether the siG12D-induced IFN-β is secreted and induces downstream signaling in an autocrine manner, the secretion of IP10 cytokine was assessed using an ELISA assay on PANC-1 (KRAS G12D mutated) and BxPc3 (KRAS wt) cells transfected with siG12D. PANC-1 cells show a clear difference between untreated and siG12D-transfected cells, the response in siG12D transfected PANC-1 cells being in the same range as that of the poly(I:C) positive control. IP-10 secretion in response to siG12D was also observed in BxPc3 cells, a pancreatic cancer cell line that is non-KRAS mutated. Similar to the response in PANC-1 cells, also in BxPc3 the siG12D and poly(I:C) treated cells showed a similar response, though to a lesser extent compared to that observed in PANC-1 cells (FIG. 2C).

Cytokine induction by siG12D was also studied in human peripheral blood mononuclear cells (PBMCs). Fresh human PBMCs were prepared by Ficoll gradient centrifugation from buffy coat (centrifuged blood, enriched for leukocytes and poor in erythrocytes) of three different (anonymous) healthy donors. siG12D was transfected in 3 different concentrations using two different transfection reactions (Dotap and Geneporter-2). Positive and negative controls were used. Human PBMCs comprise a mixed leukocyte population (monocytes, dendritic cells, T-lymphocytes, B-lymphocytes and NK cells) expressing all TLRs. Cell culture supernatants were analyzed for cytokine expression by Multiplex ELISA 24 hours post transfection.

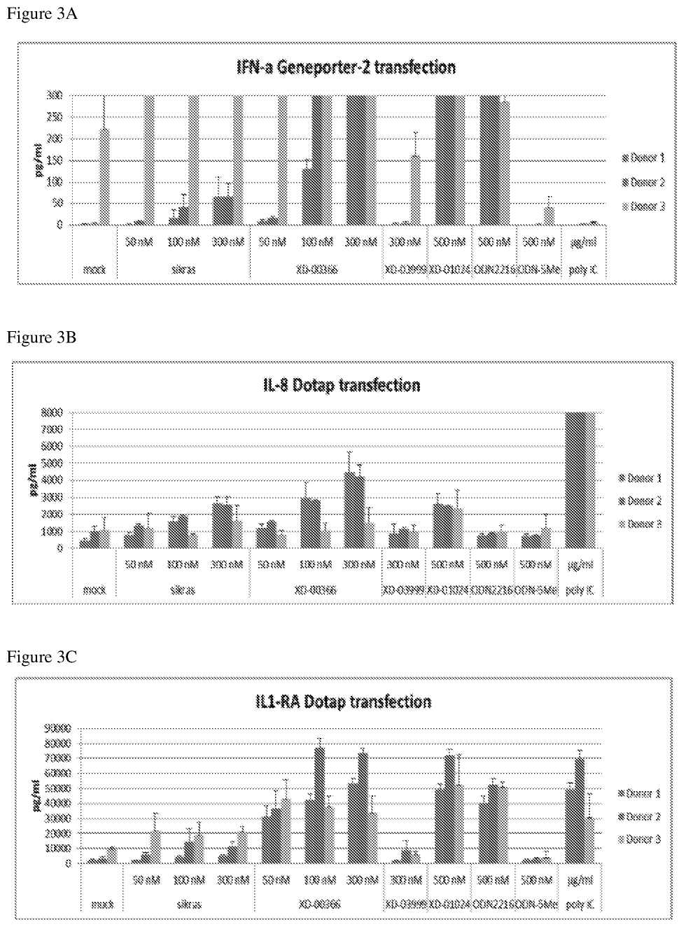

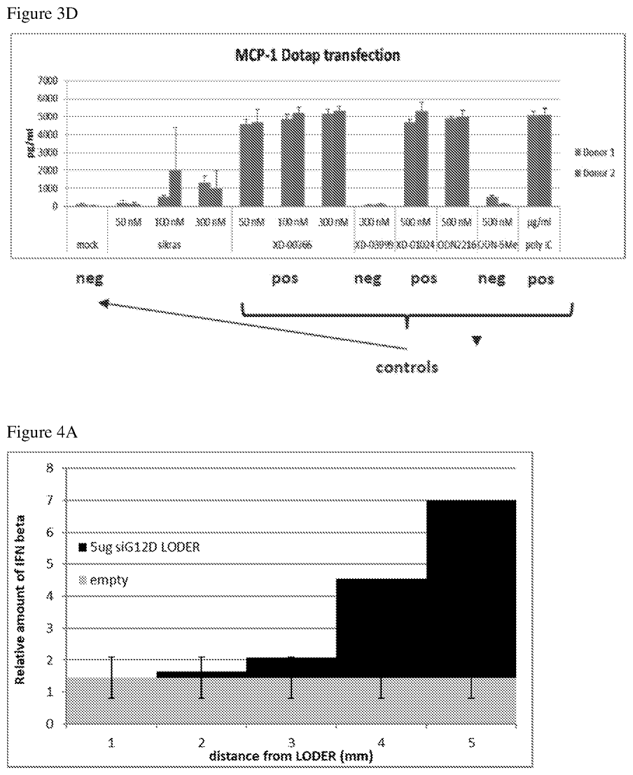

siG12D was found to induce levels of IFN-α, IL1-RA, IL-8 and MCP-1, in a dose response, with an almost negligible effect observed at a dose of 50 nM. The response observed at all levels was weaker than with the positive control siRNA (25mer blunt end) (FIG. 3).

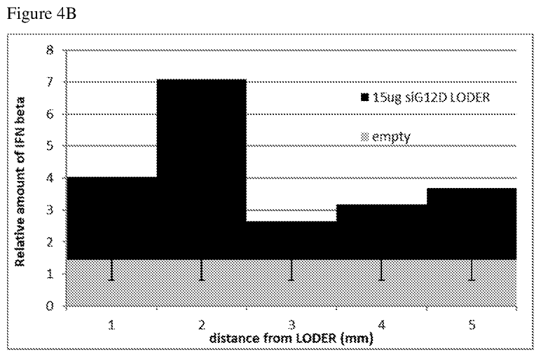

In addition to the in vitro experiments described above, an in vivo experiment was also conducted to assess the potential for innate immune system induction by siG12D-LODER. Tumor xenografts were established by subcutaneous injection of 10⁶ viable Panc 02 cells (in 1504, PBS), in log-phase growth into the flanks of C57BL/6 mice. When tumors measured an average volume of 80 mm³, mice were divided into equal groups according to volume. siG12D-LODERs or empty LODERs were implanted into tumors under anesthesia (one LODER/tumor). The LODERs used for implantation in mice were of the same type as described above in Example 1. Tumor growth was followed by caliper measurement. To explore the pattern of IFNβ induction in tumor tissue following treatment with siG12D-LODER, we detected the relative levels of IFN-β in the surrounding tumor tissue as a function of radial distance from the LODER border. tumors were formaline fixed and paraffin embedded (FFPE), sectioned, and tissue was scraped from slides for RNA purification. Gene expression was quantified by RT-PCR. Tumor tissue sections were scraped radially in concentric rings of 1 mm width, at 1, 2, 3, 4 and 5 mm radial distance from the LODER. The results reveal that siG12D-LODER induces IFN-β mostly in the border of the tumor at the low siG12D dose of 5 μg, suggesting that in tissue, macrophages are involved in the immune induction. In tumors treated with the high dose of 15 μg siG12D/LODER, induction of IFN-β was observed throughout the tumor (FIG. 4).

### Example 3: Effect of siG12D-LODER on T-Cell Infiltration into Tumors In Vivo

Example 1 describes how the siG12D-LODER increases tumor necrosis and void volume. This example demonstrates that this effect of siG12D-LODER also enhances the infiltration and distribution of T cells within the tumor in a syngeneic, orthotopic pancreatic cancer model. Accordingly, this example shows that a siRNA-delivering LODER can condition (i.e. enhance the efficacy) of immunotherapy in a solid tumor.

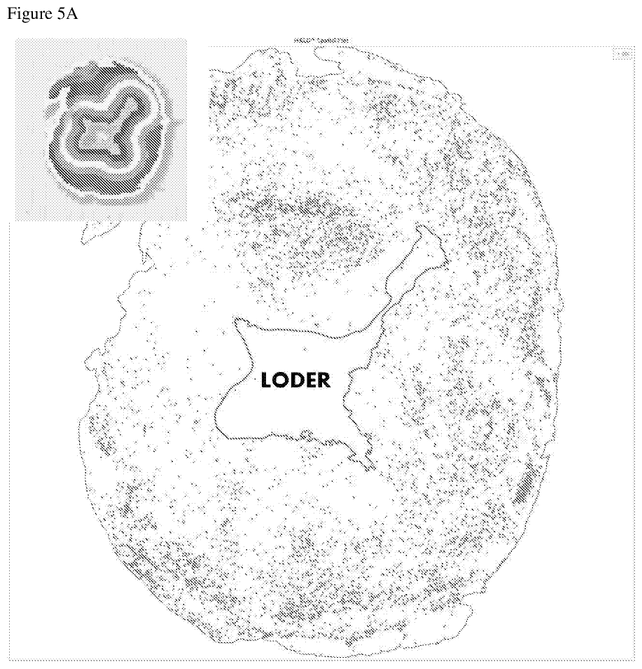

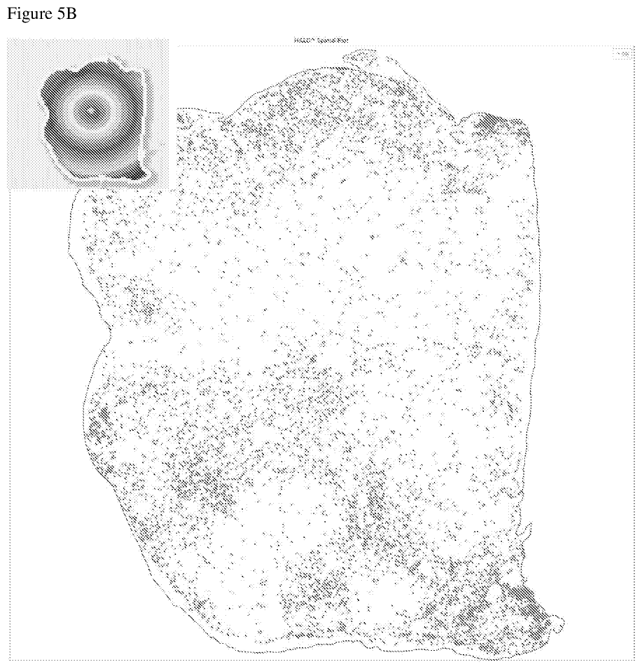

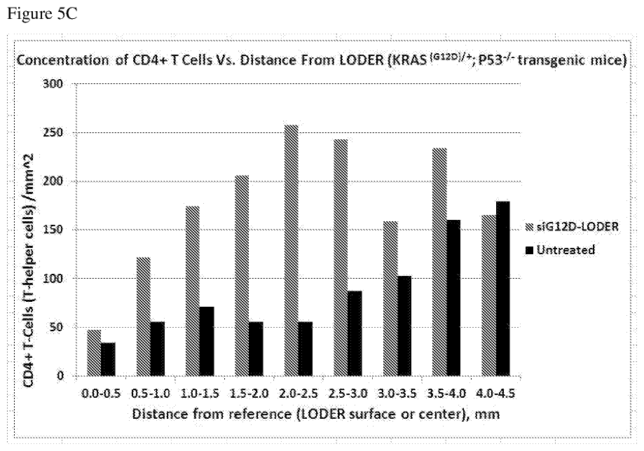

Pancreatic tumor allografts from Pdx1-Cre; LSL-KRAS^{(G12D)/+}; P53^{-/-} transgenic mice were maintained subcutaneously in C57BL/6 mice before orthotopic implantation. When the volume of seed tumor reached 700-1000 mm³, tumors were collected and cut into pieces of about 4 mm in diameter. A siG12D-LODER was inserted into each tumor piece for the treated group. The siG12D-LODERs implanted were of similar characteristics to the LODERs described above in Example 1, but with a diameter of ˜0.8 mm. For the untreated group, the tumor piece was sham operated. The tumor pieces with or without LODER were sewed into the pancreas of C57BL/6 mice. Two weeks after tumor inoculation mice were killed, and tumors were taken for histological analysis. FIGS. 5A-5C show a comparison between a treated and an untreated sample for CD4 staining (identifying CD4+ T cells). Cells positive for CD4 staining were counted at varying distances from the LODER edge in the siG12D-LODER treated tumor. In the untreated tumor, CD4-positive cells were counted at varying distances from the tumor center. This was done by counting positively stained cells within 0.5 mm-wide concentric rings around the LODER edge/tumor center. All cells within each concentric ring were counted and the positively-stained cell concentration was calculated. Positively-stained cells are shown in FIGS. 5A and 5B in the treated and untreated sample, respectively. The quantitated and compared results presented in FIG. 5C show a higher concentration of CD4+ T cells in the middle of the tumor following siG12D-LODER treatment.

### Example 4: Effect of siG12D-LODER on T-Cell Infiltration into Tumors In Vivo

This example will demonstrate the effect of siG12D-LODER on the infiltration and distribution of T cells within the tumor in a syngeneic, subcutaneous tumor model.

The experiment will test the effect of siG12D-LODER on immune cell infiltration into a solid tumor (T cells, macrophages and natural killer cells), as well as other immune-related effects. The CT26 mouse colon carcinoma cell line is commonly used in drug development and has also been used in the development of several approved immuno-oncology drugs (ipilimumab (Yervoy), nivolumab (Opdivo), atezolizumab (Tecentriq)).

CT26 cells are KRAS G12D-mutated and were previously shown in vitro by Silenseed to be affected by siG12D treatment (reduction of cell survival). Therefore, the CT26 cell line may serve as a good model for testing the effect of siG12D-LODER treatment on immune cell infiltration in a solid tumor.

CT26 cells will be injected subcutaneously into the right flank of Balb/c mice. When tumors reach a minimum size of ˜80 mm³ (˜5.5-6 mm diameter, measured by caliper), mice will be divided into groups of similar average tumor size for the following treatments: (1) Untreated (6 mice); (2) Empty LODER (6 mice); and (3) siG12D-LODER (12 μg) (6 mice). The LODERs used will be of the same characteristics described above, with a diameter of ˜0.8 mm.

One week after LODER implantation, mice will be killed and tumors will be taken for the following analyses: Tumor size, and histology. For histological analysis tumors will be cut into two halves. LODER-implanted tumors will be cut in such a way that the plane of the cut will pass through the LODER. Tissue and cellular structure will be observed by standard H&E staining. Immunohistochemistry staining will be used for detection of KRAS/KRAS G12D, CD3, CD4, CD8, FoxP3, F4/80, CD335, and IFNg.

It is expected that as in the previous examples, samples from siG12D-LODER-treated tumors will show a greater amount of immune cell infiltration than the untreated tumors.

### Example 5: Effect of siG12D on Infiltration of T-Cells and NK Cells In Vitro

This example will demonstrate the influence of siG12D on T-cell and NK cell migration into a tumor culture in a multi-chamber cell migration model.

PANC-1 cells are seeded to 80% confluency, and on the next day, transfected using Lipofectamine 2000 with: siG12D, poly (I:C), and mock transfection,

One day post-transfection, cells are detached, counted, and seeded in the lower chamber of a transwell plate with an 8 μm pore size and grown to confluence. The upper chamber of each plate is loaded with either Jurkat T-cells or CD8+ T-cells or NK cells (2×10⁵ Jurkat cells were loaded in a 6-well transwell plate (Del Galdo and Jimenez 2007)).

The transwell plates are then incubated for 3 hours or 6 hours; and the lower chambers are photographed at each time point (6 random fields at 200× magnification). The Jurkat and/or CD8+ T-cells and/or NK cells that had migrated into the lower chamber are counted or assessed by a colorimetric assay. Each experiment will be done in triplicate.

### Example 6: Clinical Study of Immunotherapy+/−siG12D-LODER

This example will show the effect of combining immunotherapy with ultrasound-guided endoscopic administration of siG12D-LODER.

In this study pancreatic cancer patients will be provided with immunotherapeutic agents (e.g. nivolumab, ipilimumab, or pembrolizumab), with or without additional chemotherapy agent (FOLFIRINOX, Gemcitabine+/−nab-paclitaxel). Patients will be followed for two years to measure tumor overall response rate (ORR), as a primary endpoint and for progression free survival (PFS) and for overall survival.

There are many possible embodiments for the scheduling of administration of each of the drug types. For example the drugs can be provided as adjuvant or as neoadjuvant. In one embodiment the siG12D-LODER is provided at first (e.g. following screening), then one-two weeks later the IO agent is administrated, with/without chemotherapy.

In another embodiment the patient already is receiving systemic treatment of IO or chemotherapy or combination, and the siG12D-LODER is provided in addition.

Patient staging is not limited to a specific stage. For example, Locally Advanced Pancreatic Cancer (LAPC, stage III) can be the target population. However, in another embodiment Stage IV (metastatic) can be included. In another embodiment all stages from I to IV can be included in the inclusion criteria.

siG12D-LODER for clinical use in human is 5.5±1.0 mm long and with a diameter of 0.80±0.04 mm, of the same PLGA matrix described above, with siG12D embedded in the polymeric matrix.

Age and gender of patient is not limited. In one embodiment an age range of 18 years to 76 years meets the inclusion criteria.

Doses, of each of the drugs, are not limited. In one embodiment the patient receives administration of 6 LODERS every 3 months (˜2000n of siG12D per administration).

### REFERENCES

Brahmer, J R. Clin Adv Hematol Oncol. 10(10):674-5; 2012.
Deshayes S et al., Biochim Biophys Acta. 1798(12):2304-14; 2010
Feig C et al., Clin Cancer Res. 18(16):4266-76; 2012.
Feig C et al., Proc Natl Acad Sci USA. 110(50):20212-7; 2013.
Javle M et al., Cancer Treatment Reviews. 44:17-25; 2016.
Del Galdo F & Jimenez S A, Arthritis Rheum. 56(10):3478-88; 2007.
Golan T et al., Oncotarget. 6 (27): 24560-70; 2015.
Liu S Y & Wu Y L., J Hematol Oncol. 10(1):136; 2017.
Makadia and Siegel, Polymers 2011, 3:1377-1397.
Pooga M et al., FASEB J. 12(1):67-77; 1998.
Ramot Y et al., Toxicologic Pathology. 44(6):856-65; 2016.
Royal R E et al., J Immunother. 33 (8): 828-33; 2010.
Shemi A et al., Oncotarget. 6 (37):39564-77; 2015.
Zorde Khvalevsky E et al., Proc Natl Acad Sci. 110 (51): 20723-8; 2013.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for conditioning a solid tumor in a subject to immunotherapy treatment, comprising:
administering to a subject in need there of a polymeric drug delivery device comprising a chemotherapeutic agent,
wherein delivery of the chemotherapeutic agent to the subject conditions the solid tumor to the immunotherapy treatment.

2. The method of claim 1, wherein the polymeric drug delivery device comprises a biocompatible polymeric matrix comprising a polymer selected from the group consisting of poly(glycolide-co-lactide) (PLGA), polylactic acid (PLA), polyglycolic acid (PGA), polyethylene glycol (PEG), and polycaprolactone (PCL), with or without additives including cryoprotectants.

3. The method of claim 1, wherein the chemotherapeutic agent is a nucleic acid, peptide, small molecule, antibody or fragment thereof, or a combination thereof.

4. The method of claim 3, wherein the nucleic acid comprises a single-stranded RNA or a double stranded RNA interference (RNAi) agent.

5. The method of claim 4, wherein the RNAi agent is KRAS siG12D.

6. The method of claim 1, wherein the polymeric drug delivery device is a LODER comprising 75-90% PLGA (85:15); 5-15% mannitol, and 0.1-0.5% sodium bicarbonate.

7. The method of claim 1, further comprising an immunotherapy composition.

8. The method of claim 7, wherein the immunotherapy composition is selected from the group consisting of a PD-1 inhibitor, a PD-L1 inhibitor, and a CTLA-4 inhibitor.

9. The method of claim 8, wherein the PD-1 inhibitor is Pembrolizumab (Keytruda) or Nivolumab (Opdivo), or a biosimilar thereof; wherein the PD-L1 inhibitor is Atezolizumab (Tecentriq), Avelumab (Bavencio), or Durvalumab (Imfinzi), or biosimilars thereof; and wherein the CTLA-4 inhibitor is ipilimumab (Yervoy), or a biosimilar thereof.

10. A method for treatment of a solid tumor comprising:
administering to a subject in need thereof a polymeric drug delivery device comprising a chemotherapeutic agent; and
an immunotherapy composition.

11. The method of claim 10, wherein the polymeric drug delivery device comprises a biocompatible polymeric matrix, comprising a polymer selected from the group consisting of poly(glycolide-co-lactide) (PLGA), polylactic acid (PLA), polyglycolic acid (PGA), polyethylene glycol (PEG), and polycaprolactone (PCL)), with or without additives including cryoprotectants.

12. The method of claim 10, wherein the chemotherapeutic agent is a nucleic acid, peptide, small molecule, antibody or fragment thereof, or a combination thereof.

13. The method of claim 12, wherein the nucleic acid comprises a single-stranded RNA or a double stranded RNA interference (RNAi) agent.

14. The method of claim 13, wherein the RNAi agent is KRAS siG12D.

15. The composition method of claim 10, wherein the polymeric drug delivery device is a LODER comprising 75-90% PLGA (85:15); 5-15% mannitol, and 0.1-0.5% sodium bicarbonate.

16. The method of claim 11, wherein the solid tumor is a pancreatic tumor.

17. The composition method of claim 11, wherein the immunotherapy composition is at least one of a PD-1 inhibitor, PD-L1 inhibitor, or CTLA-4 inhibitor.

18. The method of claim 17, wherein the PD-1 inhibitor is Pembrolizumab (Keytruda) or Nivolumab (Opdivo), or a biosimilar thereof; wherein the PD-L1 inhibitor is Atezolizumab (Tecentriq), Avelumab (Bavencio), or Durvalumab (Imfinzi), or biosimilars thereof; and wherein the CTLA-4 inhibitor is ipilimumab (Yervoy), or a biosimilar thereof.


SEQUENCE LISTING (15 sequences)

SEQ ID NO: 1 - DNA, 21 bp, Artificial Sequence
  Synthetic oligonucleotide
  misc_feature (1)..(19): RNA
  misc_feature (20)..(21): DNA
guuggagcugauggcguagtt

SEQ ID NO: 2 - DNA, 21 bp, Artificial Sequence
  Synthetic oligonucleotide
  misc_feature (1)..(19): RNA
  misc_feature (20)..(21): DNA
cuacgccaucagcuccaactt

SEQ ID NO: 3 - DNA, 21 bp, Artificial Sequence
  Synthetic oligonucleotide
  misc_feature (1)..(19): RNA
  misc_feature (20)..(21): DNA
ugccagggaccauguuuuguu

SEQ ID NO: 4 - DNA, 21 bp, Artificial Sequence
  Synthetic oligonucleotide
  misc_feature (1)..(19): RNA
  misc_feature (20)..(21): DNA
caaaacauggucccuggcauu

SEQ ID NO: 5 - DNA, 21 bp, Artificial Sequence
  Synthetic oligonucleotide
  misc_feature (1)..(19): RNA
  misc_feature (20)..(21): DNA
augaauggaaccagcaacatt

SEQ ID NO: 6 - DNA, 21 bp, Artificial Sequence
  Synthetic oligonucleotide
  misc_feature (1)..(19): RNA
  misc_feature (20)..(21): DNA
uguugcugguuccauucautt

SEQ ID NO: 7 - DNA, 21 bp, Artificial Sequence
  Synthetic oligonucleotide
  misc_feature (1)..(19): RNA
  misc_feature (20)..(21): DNA
aagaccaaccgauggaggatt

SEQ ID NO: 8 - DNA, 21 bp, Artificial Sequence
  Synthetic oligonucleotide
  misc_feature (1)..(19): RNA
  misc_feature (20)..(21): DNA
uccuccaucgguuggucuutt

SEQ ID NO: 9 - DNA, 21 bp, Artificial Sequence
  Synthetic oligonucleotide
  misc_feature (1)..(1): 2′-O-methyl modified residue
  misc_feature (1)..(19): RNA
  misc_feature (3)..(3): 2′-O-methyl modified residue
  misc_feature (5)..(5): 2′-O-methyl modified residue
  misc_feature (7)..(7): 2′-O-methyl modified residue
  misc_feature (9)..(9): 2′-O-methyl modified residue
  misc_feature (11)..(11): 2′-O-methyl modified residue
  misc_feature (13)..(13): 2′-O-methyl modified residue
  misc_feature (15)..(15): 2′-O-methyl modified residue
  misc_feature (17)..(17): 2′-O-methyl modified residue
  misc_feature (19)..(19): 2′-O-methyl modified residue
  misc_feature (20)..(21): DNA
guuggagcugauggcguagtt

SEQ ID NO: 10 - DNA, 21 bp, Artificial Sequence
  Synthetic oligonucleotide
  misc_feature (1)..(19): RNA
  misc_feature (2)..(2): O-methyl modified residue
  misc_feature (4)..(4): O-methyl modified residue
  misc_feature (6)..(6): O-methyl modified residue
  misc_feature (8)..(8): O-methyl modified residue
  misc_feature (14)..(14): O-methyl modified residue
  misc_feature (16)..(16): O-methyl modified residue
  misc_feature (18)..(18): O-methyl modified residue
  misc_feature (20)..(21): DNA
cuacgccaucagcuccaactt

SEQ ID NO: 11 - protein, 11 aa, Artificial Sequence
  Synthetic peptide
YGRKKRRQRRR

SEQ ID NO: 12 - protein, 17 aa, Artificial Sequence
  Synthetic peptide
GALFLGFLGAAGSTMGA

SEQ ID NO: 13 - protein, 12 aa, Artificial Sequence
  Synthetic peptide
KETWWETWWTEW

SEQ ID NO: 14 - DNA, 21 bp, Artificial Sequence
  Synthetic oligonucleotide
  misc_feature (1)..(19): RNA
  misc_feature (20)..(21): DNA
cuuacgcugaguacuucgatt

SEQ ID NO: 15 - DNA, 21 bp, Artificial Sequence
  Synthetic oligonucleotide
  misc_feature (1)..(19): RNA
  misc_feature (20)..(21): DNA
ucgaaguacucagcguaagtt